(19) United States
(12) Patent Application Publication
(10) Pub. No.: US 2025/0107840 A1
(43) Pub. Date: Apr. 3, 2025

(54) ANTI-STICK COATINGS FOR SURGICAL TOOLS

(71) Applicant: Cilag GmbH International, Zug (CH)

(72) Inventors: Luling Wang, Mason, OH (US); Gregory A. Trees, Cincinnati, OH (US); Candice Otrembiak, Loveland, OH (US); John Michael Sarley, Mason, OH (US)

(21) Appl. No.: 18/896,213

(22) Filed: Sep. 25, 2024

Related U.S. Application Data
(60) Provisional application No. 63/586,229, filed on Sep. 28, 2023.

(51) Int. Cl. A61B 18/14; A61B 18/00

(52) U.S. Cl. CPC A61B 18/1445; A61B 2018/00077; A61B 2018/00136; A61B 2018/00702

(57) ABSTRACT

Various exemplary devices, systems, and methods for anti-stick coatings for surgical tools are provided. In general, a method of manufacturing a surgical tool includes forming, using plasma enhanced chemical vapor deposition with hexamethyldisiloxane as a precursor material, a coating on a conductive tissue treating surface of an end effector, the coating comprising a silicone material, wherein the coatings are effective to prevent tissue sticking to the jaws during an electrosurgical procedure.

### RELATED APPLICATION

This application claims priority under 35 U.S.C. § 119 (e) to U.S. Provisional Application No. 63/586,229 filed Sep. 28, 2023, the entire contents of which are hereby expressly incorporated by reference herein.

### FIELD

The present disclosure generally relates to anti-stick coatings for surgical tools.

### BACKGROUND

Various types of surgical devices are used for grasping, cutting, and sealing tissue. In general, these devices have jaws configured to grasp tissue and a cutting mechanism configured to be advanced through the tissue to sever it. Some of these devices can apply energy to the tissue disposed between the jaws to promote hemostasis. In instances where the tissue includes a vessel, these devices can also apply energy to the tissue disposed between the jaws to seal the vessel.

A common issue when using one of these surgical devices is that the jaws, or other components of the surgical device, stick to the tissue that the device is grasping, cutting, sealing, or otherwise treating. Tissue stuck to a surgical device may be pulled out of place and/or damaged when removing the surgical device from the tissue. In some instances, this damage may compromise the treatment performed by the surgical device.

Attempts have been made in the art to provide surgical devices that include various non-stick surface coatings. For example, U.S. Pat. Nos. 10,709,497 and 10,973,569, both assigned to Covidien LP, discuss various polydimethylsiloxane non-stick coatings. However, these patents teach that if the applied non-stick coating is too thin (e.g., less than about 20 nm), the non-stick coating may not provide adequate tissue sticking reduction, and if the non-stick coating is above a particular thickness (e.g., greater than about 200 nm) this may create a uniform dielectric barrier or surface impedance on the sealing plates.

Accordingly, there remains a need for improved devices, systems, and methods for reducing sticking between surgical tools and tissue these tools contact.

### SUMMARY

In general, devices, systems, and methods for anti-stick coatings for surgical tools are provided.

In one aspect, a method of manufacturing a surgical tool is provided. In one embodiment, the method includes forming, using plasma enhanced chemical vapor deposition with a precursor material, a coating on a first conductive tissue treating surface of an end effector of an electrosurgical tool and on a second conductive tissue treating surface of the end effector of the electrosurgical tool, the coating comprising a silicone material that is effective to prevent tissue sticking to the conductive tissue treating surface during an electrosurgical sealing procedure, wherein the coating has a thickness between 7-17 nm or 220 and 300 nm. In some embodiments, the surgical tool is configured to deliver approximately 20 to 210, such as approximately 60 to 175, joules of energy to the tissue.

The method can vary in any number of ways. For example, the precursor material can be hexamethyldisiloxane.

For another example, the silicone material can be a polydimethylsiloxane-like material.

For still another example, the silicone material can include polydimethylsiloxane.

For another example, the coating can have a thickness of approximately 15 nm.

For yet another example, the coating can have a thickness between approximately 220 and 300 nm.

For another example, the coating can have a thickness between approximately 7 and 17 nm.

For still another example, the method can also include creating gaps or holes in the coating.

For yet another example, the method can also include, after forming the coating, assembling the end effector using the first jaw component and a second jaw component.

According to another aspect, an end effector of an electrosurgical device is disclosed. In one embodiment, the end effector includes a first jaw component including first conductive tissue treating surface, a second jaw component operatively coupled to the first jaw component, the second jaw component having a second conductive tissue treating surface, and a coating on the first conductive tissue treating surface and the second conductive tissue treating surface, the coating comprising a silicone-based material having a thickness between 7 and 17 nm or 220 and 300 nm. In some embodiments, the end effector is configured to deliver approximately 20 to 210 joules, such as approximately 60 to 175, joules of energy to tissue. In some embodiments, the vessel sealer delivers between approximately 40 to 210 joules of energy to tissue or between approximately 20 to 180 joules of energy to tissue. Examples include approximately 20, 30, 40, 50, 60, 70, 80, 90, 100, 110, 120, 130, 140, 150, 160, 170, 180, 190, 200, and 210 joules of energy to tissue.

The end effector can vary in any number of ways. For example, the silicone material can be derived from hexamethyldisiloxane.

For another example, the silicone material can be a polydimethylsiloxane-like material.

For yet another example, the silicone material can include polydimethylsiloxane.

For another example, the coating can have a thickness of approximately 15 nm.

For still another example, the coating can have a thickness between 7 and 17 nm.

For still another example, the coating can have a thickness between 220 and 300 nm.

For still another example, the first jaw component and the second jaw component can have a curved shape.

For yet another example, the first jaw component and the second jaw component have a straight shape.

### BRIEF DESCRIPTION OF THE DRAWINGS

This disclosure will be more fully understood from the following detailed description taken in conjunction with the accompanying drawings, in which:

FIG. 1 is a schematic cross-sectional view of a portion of a surgical tool with an anti-stick coating;

FIG. 2 is a perspective view of an exemplary surgical tool coated with the anti-stick coating of FIG. 1;

FIG. 3 is a perspective view of the end effector of the surgical tool of FIG. 2;

FIG. 4 is a schematic of a process for forming the coating of FIG. 1 on the surgical tool of FIG. 2;

FIG. 5A is a schematic of a plasma enhanced chemical vapor deposition system for creating a first layer of the coating of FIG. 1;

FIG. 5B is a flow chart showing a process for creating a first layer of the coating of FIG. 1; and

FIG. 5C is a Fourier Transform Infrared spectroscopy output showing the spectrum of a PDMS-like material of the coating of FIG. 1.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices, systems, and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Further, in the present disclosure, like-named components of the embodiments generally have similar features, and thus within a particular embodiment each feature of each like-named component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. Sizes and shapes of the systems and devices, and the components thereof, can depend at least on the anatomy of the subject in which the systems and devices will be used, the size and shape of components with which the systems and devices will be used, and the methods and procedures in which the systems and devices will be used.

Various exemplary devices, systems, and methods for manufacturing anti-stick coatings for surgical tools are provided. In general, during a surgical procedure, a surgical tool contacts tissue to manipulate and/or repair and/or coagulate the tissue. During or after manipulation and/or repair and/or coagulation of tissue, the surgical tool is separated from the tissue. The devices and methods described herein provide the ability to separate the surgical tool from the tissue without damaging the tissue and/or moving the tissue out of position due to sticking. The anti-stick coating includes a silicone material or a silicone-like material and is applied to an area of the surgical tool, or its components prior to assembly, using a plasma enhanced chemical vapor deposition process.

The systems, devices, and methods described herein have applicability in a variety of surgical procedures that require energy to be delivered to the tissue including vessel repair and/or coagulation, soft tissue repair and/or coagulation, and/or tissue manipulation, such as laparoscopic procedures using advanced bipolar RF energy devices, ultrasonic laparoscopic surgical devices, or open surgery using monopolar devices.

During surgical procedures, especially electrosurgical procedures, tissue sticking to surgical tools used to perform the surgery can cause interruption of the procedure and sometimes can cause damage to the tissue. Therefore, it is important that the surgical tools used during the procedure not stick to the tissue being treated. Anti-stick coatings applied to surgical tools, for example, on conductive tissue-treating surfaces of electrosurgical tools, such as the anti-stick coatings discussed herein, can prevent tissue from sticking to surgical tools. It is also important that the anti-stick coating not impair the functionality of the surgical tools, for example, the ability to deliver energy to tissue to seal/coagulate the tissue. The anti-stick coatings discussed herein allow for electrosurgical energy to pass therethrough to enable effective electrosurgical treatment.

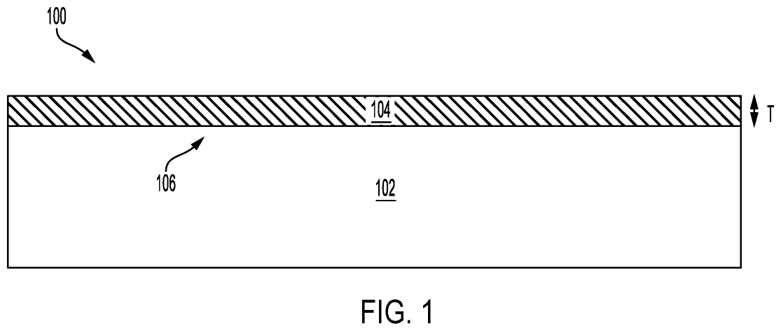

FIG. 1 is a cross-sectional view of a portion 100 of a surgical tool 102 with an anti-stick coating 104 that is formed on a conductive, tissue treating surface 106 of the surgical tool 102. In some embodiments, the tissue treating surface is a conductive tissue treating surface configured to transmit electrical energy to tissue during an electrosurgical procedure.

In the embodiment of FIG. 1, the coating 104 is formed of a silicone or silicone-like material. In some embodiments, the silicone material is a polydimethylsiloxane-like material that is derived from hexamethyldisiloxane. In a preferred embodiment, the coating 104 has a thickness that is between approximately 7 and 17 nm, preferably approximately 15 nm. In some embodiments, the coating has a thickness T that is approximately 7, 8, 9, 10, 11, 12, 13, 14, 15, 16, or 17 nm. In another preferred embodiment, the coating 104 has a thickness T that is between approximately 220 and 300 nm. In some embodiments, the coating 104 has a thickness T that is approximately 220, 230, 240, 250, 260, 270, 280, 290, or 300 nm. In some embodiments, the coating 104 has a thickness T of between approximately 7 and 20 nm or between approximately 200 and 300 nm.

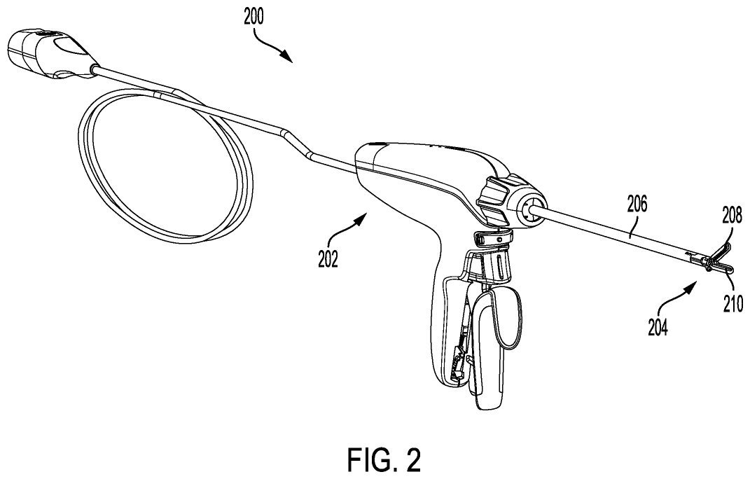

FIG. 2 is a perspective view of an exemplary surgical tool 200 coated with the anti-stick coating 104 of FIG. 1. The surgical tool 200 is an electrosurgical vessel sealing tool that is configured to grab, seal/coagulate, and cut tissue. The surgical tool 200 includes a proximal portion 202 which may be coupled to a handle for manual operation of the surgical tool or an adapter for use with a robotic surgical system for robotic operation of the surgical tool. The surgical tool 200 includes an end effector 204 at the distal end of a shaft 206. The end effector 204 includes a first jaw component 208 and a second jaw component 210. The jaws are configured to grab and hold tissue during operation of the surgical tool 200. In the embodiment of FIG. 2, a smaller volume conductive metal electrode is formed in the second jaw component 210 whereas the first jaw component 208 serves as a larger volume conductive metal electrode. In some embodiments, upper jaw and lower jaw electrodes may be more similar in volume to each other, resulting in a more thermally balanced design when delivering energy to the tissue.

In some embodiments, instead of the vessel sealer surgical tool 200 shown in FIG. 2, the surgical tool may be a surgical stapler, surgical saw, a surgical drill, or another surgical implement or tool configured to interact with tissue. One of ordinary skill in the art would understand that the anti-stick coatings described herein could be applied to any tissue-contacting tool, including any electrosurgical tool. One of ordinary skill in the art would understand that it may be advantageous to vary the thickness of the anti-stick coating based on the particular surgical tool being coated. For example, surgical tools that do not deliver electrical energy via the coated surface (e.g., a conductive tissue treating surface) may be able to tolerate a thicker anti-stick coating without compromising the performance of the surgical tool.

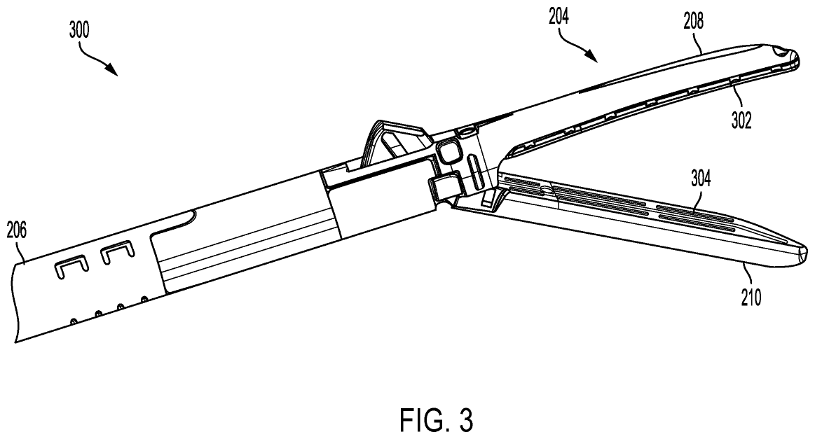

FIG. 3 is a perspective view of a distal portion 300 of the surgical tool 200 shown in FIG. 2. The surgical tool 200 is a tissue sealing tool with an end effector 204 configured to grab, seal, and cut tissue. In the embodiment of FIGS. 2 and 3, the jaws 204 have a curved shape. In some embodiments, the jaws of the end effector have a straight shape.

The distal portion 300 of the surgical tool 200 includes a shaft 206 connected to the end effector 204. The end effector 204 includes a first jaw component 208 and a second jaw component 210 that form the jaws of the end effector. The first jaw component 208 includes a first tissue treating surface 302 and the second jaw component 210 includes a second tissue treating surface 304. The first tissue treating surface 302 and the second tissue treating surface 304 are conductive surfaces. One or both of the first tissue treating surface 302 and the second tissue treating surface 304 are configured to transmit electrical energy to tissue during an electrosurgical procedure. The first and second tissue treating surfaces 302, 304 are coated with the anti-stick coating 104 shown in FIG. 1. In some embodiments, additional surfaces of the jaw components are coated with the anti-stick coating. In some embodiments, the vessel sealer delivers between approximately 20 to 210, such as approximately 60 and 175, joules of energy to tissue. In some embodiments, the vessel sealer delivers between approximately 40 to 210 joules of energy to tissue or between approximately 20 to 180 joules of energy to tissue. Examples include approximately 20, 30, 40, 50, 60, 70, 80, 90, 100, 110, 120, 130, 140, 150, 160, 170, 180, 190, 200, and 210 joules of energy to tissue.

In some embodiments, one or more of the tissue treating surfaces includes a slot configured to allow a knife to translate therein. The tissue treating surfaces may have rounded or sharp edges. The tissue treating surfaces may be generally planar or may have surface features thereon. A conductive tissue treating surface may be formed from various conductive biocompatible materials, such as metal or metal composites.

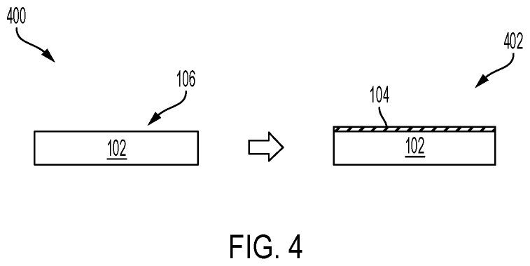

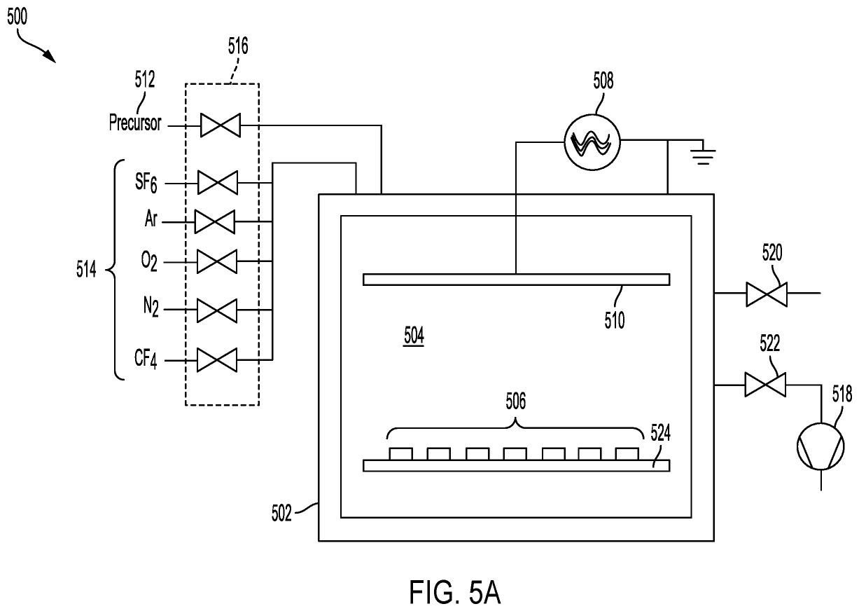

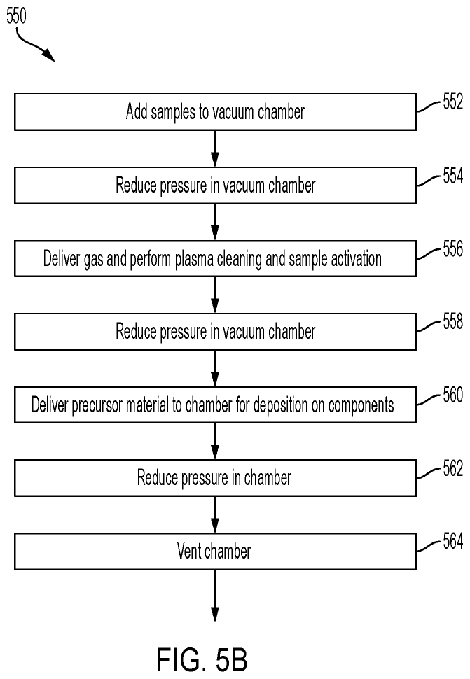

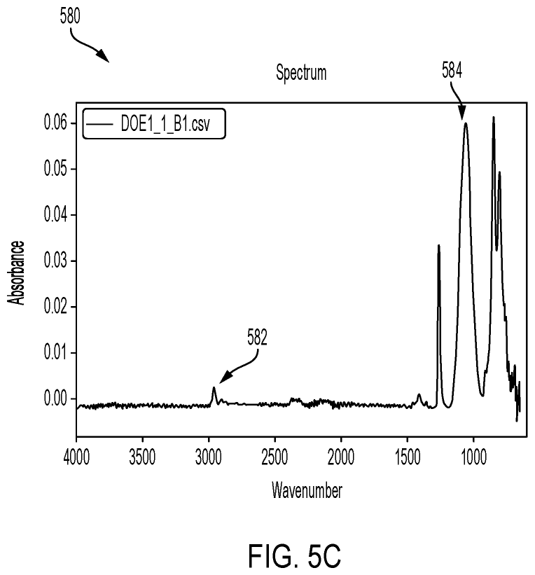

FIG. 4 is a schematic of a process of for forming the coating of FIG. 1 on the surgical tool of FIG. 2. Generally, as shown at step 400, a tissue treating surface 106 of a surgical tool 102 is exposed. In a first process, shown at step 402, the tissue treating surface 106 is coated with a coating 104. As described above with respect to FIG. 1, the coating 104 is applied using a plasma enhanced chemical vapor deposition (PECVD) process and is formed of a PDMS-like material that has a thickness of between approximately 7-17 nm or between approximately 220-300 nm. In a preferred embodiment, the thickness of the PDMS-like material is approximately 15 nm. In some embodiments, the coating can also be applied to other surface areas and/or components of the surgical tool 102 in addition to the tissue treating surface 106. An example apparatus and method for applying the coating 104 is shown in FIGS. 5A-5B.

FIG. 5A is a schematic of a system for creating the coating of FIG. 1. The plasma enhanced chemical vapor deposition system (PECVD) system 500 is used to produce polydimethylsiloxane (PDMS) like coatings on surgical tool components 506. In some embodiments, the PDMS-like coatings derived from hexamethlydisiloxane (HMDSO) are a silicone type material similar to PDMS. FIG. 5C shows Fourier Transform Infrared (FTIR) spectroscopy output for the PDMS-like coating 582, and 584. One of skill in the art would understand that other chemical precursors may be used in the PECVD process to generate a similar PDMS like structure for the anti-stick coatings described herein.

In some embodiments, the surgical tool components 506 are jaws of an end effector of a vessel sealer, for example, the end effector 204 of the surgical tool 200 shown in FIGS. 2-3. The PECVD system includes a vacuum chamber 502 that is configured to be filled with gas and plasma 504. In some embodiments, the plasma is an oxygen plasma for cleaning and activating the surface of the surgical tool components 506. In some embodiments, the gas includes sulfur hexafluoride, argon, oxygen, nitrogen, tetrafluoromethane, hydrogen, and/or other gases or gas combinations that can be used for plasma cleaning and activation. The pressure of the vacuum chamber 502 is controlled by a vacuum pump 518, vacuum valve 522, gas and/or precursor flow rates, and a vent valve 520. In some embodiments, the pressure of the vacuum chamber during the cleaning/activation and deposition steps is approximately between 25 mTorr and 750 mTorr. A person of skill in the art would understand that the vacuum pressure may be varied depending on the particular parameters of the PECVD system used. In one embodiment, shown in FIG. 5A, the plasma 504 is an oxygen plasma. First, one or more surgical tool components 506 is placed in the vacuum chamber 502. In some embodiments, assembled surgical tools are placed in the vacuum chamber 502 prior to coating. In some embodiments, it is preferred to coat surgical tool components rather than assembled surgical tools due to a size and/or shape of the surgical tool. The vacuum chamber 502 is connected to a liquid precursor chemical 512, which may be, for example hexamethlydisiloxane (HMDSO). The vacuum chamber 502 is connected to process gases 514 as well as the precursor chemical 512 via flow controllers 516. The vacuum chamber 502 is also connected to vacuum pump 518, vacuum valve 522, and vent valve 520 which allow for pressure control of the vacuum chamber 502.

A radiofrequency (RF) generator 508 provides suitable power for igniting and sustaining plasmas 504 between the powered electrode 510 and grounding electrode 524. The power of the RF generator depends on the particular PECVD system used. In some embodiments, the power of the RF generator is between 0 W to 1000 W with frequency of 13.56 MHz. A person skilled in the art would understand that other RF generators operating at different frequencies and power levels may be used, as long as such generators can ignite and sustain the plasma for the applications described herein. The plasma 504 interacts with the precursor chemical 512 to deposition material on the surgical tool components 506. In an embodiment where the surgical tool components 506 are jaw components of an end effector, tissue treating surfaces (e.g., tissue treating surfaces 302, 304 shown in FIG. 3) are coated with the PDMS-like coating. In some embodiments, the PDMS-like coating covers a surface area on the jaw components 208, 210 or on the end effector 204 that is larger than the surface area of the tissue treating surfaces 302, 304.

An exemplary process 550 for PECVD is shown in FIG. 5B. In a first step 552, the components or tools to be coated are placed inside. The pressure in the vacuum chamber 502 is then reduced, in a second step 554, to a predetermined pressure using the vacuum pump 518. In some embodiments, the predetermined pressure is approximately 50 mTorr to 300 mTorr. In a third step 556, after the pressure in the vacuum chamber 502 is reduced to the predetermined pressure, gas flows into the vacuum chamber 502 to reach a predetermined pressure, an oxygen plasma is used to clean and activate the surfaces (e.g., tissue treating surfaces 302, 304 of end effector 204 shown in FIG. 3) to prepare the surgical tool components 506 for coating depositions. This oxygen plasma treatment cleans and activates the sample substrate prior to deposition of the coating. In some embodiments, plasma of other gases or mixtures of gases may also be used for this purpose. In a fourth step 558, the chamber pressure is reduced again by pumping out the gases and reactive species. In a fifth step 560, the precursor chemical 512, which in the present embodiment is HMDSO, is delivered to the vacuum chamber 502 in a vapor phase. The RF generator 508 delivers RF energy to the electrode 510 to create plasma and begin the chemical deposition on the surface of the surgical tool components 506. One of skill in the art would understand that adjusting the process 550 by adding, modifying, or reducing certain steps may achieve similar results.

The deposition process is controlled by the generator power, time, flow rate, chamber pressure, and chamber temperature. In some embodiments, the chamber temperature is at approximately room temperature, for example, approximately between 18° C. to 31° C. In some embodiments, the chamber temperature is above room temperature, for example, approximately between 32° C. to 100° C. With power, flow rate, chamber pressure, and chamber temperature controlled, the coating thickness is determined based on the amount of time that the deposition process is run. For example, a coating deposition rate may be approximately 7 nm/min. For example, the overall time of the deposition process may be approximately 90 seconds to achieve a coating thickness of approximately 10 nm and may be approximately 40 minutes to achieve a coating thicknesses of approximately 250 nm. In some embodiments, during step 560, a carrier gas such as O₂ or Argon can be delivered together with HMDSO into the chamber. One of ordinary skill in the art would understand that other carrier gases may be used during the deposition step 560 to adjust the deposition rate. It is understood that the deposition rate may be adjusted based on the process parameters described above.

In a sixth step 562, after the deposition process is completed and the desired coating thickness is achieved, the pressure of the vacuum chamber 502 is reduced to remove extra material from the chamber. Depending on the precursor material and the deposition processes of step 560, a purge process may be added between the delivery and deposition step 562 and the venting step 564, wherein the gases and reactive species are pumped out to the exhaust before the venting step 564. The vacuum chamber 502 is then vented (in the sixth step 562) with N₂ or air to atmospheric pressure, which allows the surgical tool components 506 to be removed from the vacuum chamber 502. In some embodiments, after the surgical tool components 506 are coated and removed from the vacuum chamber 502, the surgical tool components 506 are assembled to form an end effector of a surgical tool (e.g., end effector 204 of surgical tool 200 shown in FIGS. 2-3). In the surgical tool 200 shown in FIGS. 2-3, the jaw components 208, 210 can be assembled to form the jaws of the end effector 204 after the PDMS-like coating is deposited. In some embodiment, the end effector 204 can be coated directly in vacuum chamber 502 with the tissue treating surfaces 304 and/or 302 exposed to plasma.

In some embodiments, the surgical tool may include a coating having gaps or holes in the coating. These gaps or holes may allow for increased transmission of electrical energy into the tissue from the conductive tissue treating surfaces. The gaps or holes may be formed by using laser ablation, chemical etching with masking, mechanical removal of selected coating areas, or combinations thereof. The gaps or holes may also be formed by using masking during the PECVD deposition steps (e.g., by covering the coated surface with a patterned mask), such that only unmasked areas are coated while masked areas remain uncoated.

As summarized below in Table 1, benchtop sticking tests were performed with vessel sealing surgical tools with a variety of coatings. Each vessel sealer (e.g., surgical tool 200 shown in FIG. 2) was activated to perform a sealing cycle which applies electrical energy to tissue. In some embodiments, the electrical energy can be delivered with the Ethicon GEN11 Generator. One of skill in the art would understand that other generators may be used for RF energy delivery. In some embodiments, the vessel sealer may deliver between approximately 20 to 210, such as approximately 60 and 175, joules to tissue during a seal cycle. In some embodiments, the vessel sealer delivers between approximately 40 to 210 joules of energy to tissue during a seal cycle or between approximately 20 to 180 joules of energy to tissue during a seal cycle. Examples include approximately 20, 30, 40, 50, 60, 70, 80, 90, 100, 110, 120, 130, 140, 150, 160, 170, 180, 190, 200, and 210 joules of energy to tissue during a seal cycle.

After the sealing cycle is complete, the jaws of the vessel sealer were opened. If the tissue fell from the device jaws upon opening without manipulation by an operator of the test, the outcome was recorded as an anti-sticking event. If the tissue did not fall from the device jaws upon opening and required some device manipulation (jaws opening, jaw closing, and/or rotation) or if the tissue required the operator to remove it by hand or with another tool, the outcome was recorded as a sticking event. Each one of these tests that includes grasping tissue, running a sealing cycle, opening the jaws, and as necessary, manipulating the vessel sealer or manually removing tissue, is considered one activation.

Table 1, below, shows results of activations on porcine tissue for two devices. Each device was an Ethicon RF Tissue Sealing device on which a large number of activations were performed, which is typical of clinical use. The first device was a device having curved jaws that was coated with a one part silicone coating by dip coating method. The one part silicone can be cured at room temperature or at an elevated temperature. The second device was a device having straight jaws that was coated with a one part silicone coating by dip coating method. The thickness of the coating on the second device was approximately 260 nm.

The curved device with the one part silicone coating had an anti-sticking rate of 0.0635. The straight device coated in one part silicone had an anti-sticking rate of 0.1110. Each of the curved devices coated with the PECVD PDMS-like material had improved performance over the one part silicone coated straight and curved devices. The third device which included a 259 nm coating, had an anti-sticking rate of 0.25.

TABLE 1
Anti-Sticking Rates

| Device | Mean | Std Dev |
|---|---|---|
| Curved + One Part Silicone | 0.0635 | 0.0091 |
| Straight + One Part Silicone | 0.1110 | 0.0016 |
| Curved + 259 nm PECVD PDMS | 0.2500 | 0.0035 |

It is important that the sealing capability of the vessel sealer be maintained even with the application of the anti-stick coating on the tissue treating surfaces of the vessel sealer. Table 2, below, shows results of burst testing on large (5-7 mm in diameter) porcine arteries which had been sealed. The porcine arteries had no tension placed on them and the test included 8 seals for 16 bursts per device. Each device was an Ethicon RF Tissue Sealer. The first device was coated with one part silicone. The second device was coated with a 259 nm thick PECVD PDMS-like coating which was formed as shown and described above with respect to FIGS. 5A-5B.

The burst pressure results for the second device, which had a coating thickness of approximately 260 nm, had similar burst pressure performance as the control device coated with one part silicone. Therefore, the PECVD applied PDMS-like anti-stick coating did not negatively affect sealing performance of the vessel sealer device.

TABLE 2
Burst Pressure (mmHg)

| Device | N | Mean | Std Dev |
|---|---|---|---|
| One part Silicone | 16 | 655.25 | 369.2 |
| 259 nm PECVD PDMS | 16 | 722.49 | 391.3 |

During the burst pressure testing shown and described with respect to Table 2 above, a total number of events that failed the pre-pressurization check were tracked for each device and are noted in Table 3, below. These events failed between 0 and 120 mmHg, but the exact point of failure is not known due to the nature of the test equipment & pre-pressurization sequence. These are considered low performing seals.

TABLE 3
Events that failed between 0 and 120 mmHg

| Device | N | Count |
|---|---|---|
| One part Silicone | 16 | 3 |
| 259 nm PECVD PDMS | 16 | 3 |

The PECVD coated device, with a coating approximately 260 nm thick, had equivalent performance as the control one part silicone device with the same number of events that failed between 0 and 120 mmHg. These results show that at least up to 259 nm coating thickness, the PDMS-like anti-stick coating did not negatively affect sealing performance of the vessel sealer device.

Table 4 below shows the benchtop sticking test results (N=3) performed on porcine tissue. A device with a 17 nm thick PECVD PDMS coating has significant improvement of the anti-sticking rates over the one-part silicone coating that was produced by dip coating in Table 1. It can be inferred that the device having the 17 nm thick PECVD PDMS coating will have satisfactory burst performance because burst performance was not negatively affected by a 259 nm thick coating, as shown in Table 3, and the 17 nm thick coating is less than one tenth as thick as the 259 nm coating.

TABLE 4
Anti-Sticking Rates

| Device | Mean | Std Dev |
|---|---|---|
| 17 nm PECVD PDMS coating | 0.48 | 0.09 |

One skilled in the art will appreciate further features and advantages of the devices, systems, and methods based on the above-described embodiments. Accordingly, this disclosure is not to be limited by what has been particularly shown and described, except as indicated by the appended claims. All publications and references cited herein are expressly incorporated herein by reference in their entirety for all purposes.

The present disclosure has been described above by way of example only within the context of the overall disclosure provided herein. It will be appreciated that modifications within the spirit and scope of the claims may be made without departing from the overall scope of the present disclosure.

What is claimed is:

1. A method of manufacturing a surgical tool, the method comprising:
forming, using plasma enhanced chemical vapor deposition with a precursor material, a coating on a first conductive tissue treating surface of an end effector of an electrosurgical tool and/or on a second conductive tissue treating surface of the end effector of the electrosurgical tool, the coating comprising a silicone material that is effective to prevent tissue sticking to the conductive tissue treating surface during an electrosurgical procedure;
wherein the coating has a thickness between 7 and 17 nm or 220 and 300 nm.

2. The method of claim 1, wherein the precursor material used for plasma enhanced chemical vapor deposition is hexamethyldisiloxane.

3. The method of claim 1, wherein the silicone material is a polydimethylsiloxane-like material.

4. The method of claim 1, wherein the silicone material comprises polydimethylsiloxane.

5. The method of claim 1, wherein the coating has a thickness of approximately 15 nm.

6. The method of claim 1, wherein the coating has a thickness between approximately 220 and 300 nm.

7. The method of claim 1, wherein the coating has a thickness between approximately 7 and 17 nm.

8. The method of claim 1, further comprising creating gaps or holes in the coating.

9. The method of claim 1, further comprising, after forming the coating, assembling the end effector using a first jaw component and a second jaw component.

10. The method of claim 1, wherein the surgical tool is configured to deliver approximately 20 to 210 joules of energy to the tissue.

11. An end effector of an electrosurgical device, comprising:
a first jaw component having a first conductive tissue treating surface;
a second jaw component operatively coupled to the first jaw component, the second jaw component having a second conductive tissue treating surface; and
a coating on the first conductive tissue treating surface and the second conductive tissue treating surface, the coating comprising a silicone-based material having a thickness between 7 and 17 nm or 220 and 300 nm.

12. The end effector of claim 11, wherein the silicone material is derived from hexamethyldisiloxane.

13. The end effector of claim 11, wherein the silicone material is a polydimethylsiloxane-like material.

14. The end effector of claim 11, wherein the silicone material comprises polydimethylsiloxane.

15. The end effector of claim 11, wherein the coating has a thickness of approximately 15 nm.

16. The end effector of claim 11, wherein the coating has a thickness between approximately 7 and 17 nm.

17. The end effector of claim 11, wherein the coating has a thickness between approximately 220 and 300 nm.

18. The end effector of claim 11, wherein the first jaw component and the second jaw component have a curved shape.

19. The end effector of claim 11, wherein the first jaw component and the second jaw component have a straight shape.

20. The end effector of claim 11, wherein the end effector is configured to deliver approximately 20 to 210 joules of energy to tissue.
